(19) United States
(12) Patent Application Publication
(10) Pub. No.: US 2023/0338002 A1
(43) Pub. Date: Oct. 26, 2023

(54) IMAGING SYSTEM AND DEVICE FOR BREAST CANCER DETECTION

(71) Applicant: VAYYAR IMAGING LTD., YEHUD (IL)

(72) Inventors: SHAHAR RIVEL, YEHUD (IL); YUVAL LOMNITZ, HERZELIYA (IL); EITAN SHARIF, KIBBUTZ GESHER-HAZIV (IL); NAFTALI CHAYAT, KFAR SABA (IL)

(21) Appl. No.: 17/908,574

(22) PCT Filed: Mar. 2, 2021
(86) PCT No.: PCT/IB2021/051727

§ 371 Date: Sep. 1, 2022

Related U.S. Application Data
(60) Provisional application No. 62/983,781, filed on Mar. 2, 2020.

(51) Int. Cl. A61B 8/08; A61B 5/00

(52) U.S. Cl. CPC A61B 8/0825; A61B 5/708

(57) ABSTRACT

An imaging system and a device for breast cancer detection are disclosed. The device includes a plurality of antennas arranged to form a polyhedral cup shape against which the breast is pressed. The antennas are mounted flat circuit boards which comprise the antennas and RF transceiver RFICs and are configured to hinge together to form a multichip module. The polyhedral shape allows antennas to be in direct contact with the breast skin without any intermediate medium. This enables dielectric constant of antennas matched with that of the breast skin or tissues thereby reducing reflections from skin-fat boundary.

### CROSS-REFERENCE TO RELATED APPLICATIONS

This application claims the benefit of priority from U.S. Provisional Patent Application No. 62/983,781, filed Mar. 2, 2020 which is incorporated by reference in its entirety.

FIELD OF THE DISCLOSURE

The disclosure herein relates to an imaging system and a device for detecting breast cancer. In particular, the disclosure relates to tight antenna array based breast cancer imaging devices, systems, and methods thereof.

### BACKGROUND

One of the worrisome and fast spreading diseases in women is Breast cancer, a major reason for female mortality nowadays. Breast cancer is a type of cancer that develops from the breast tissue. Signs of breast cancer may include a lump in the breast, a change in breast shape, dimpling of the skin, and fluid coming from the nipple, a newly-inverted nipple, or a red or scaly patch of skin. Worldwide breast cancer is one of the leading causes of death among women. A study revealed, in the USA, tens of thousands of deaths are reported yearly because of this type of cancer. 26 percent of all types of cancers among women are breast cancer. About one in eight U.S. women (about 12%) develops invasive breast cancer over the course of her lifetime. In 2020, an estimated 276,480 new cases of invasive breast cancer are expected to be diagnosed in women in the U.S., along with 48,530 new cases of non-invasive (in situ) breast cancer.

When cells in the breast tissue begin to divide and grow abnormally, the breast cancer starts. It begins with a lump or a mass and if untreated, it spreads to the entire tissue. Hence, it is very important to screen or identify lesions in the tissue at early stage. There are different technologies for examination of the breasts such as Mammogram, Ultrasound, Biopsy, and Magnetic Resonance Imaging (MRI). The Mammogram involving X-ray Mammogram has been one of the golden standards in breast cancer screening. It uses low energy X-rays to examine the human breast for diagnosis and screening. The conventional X-ray mammogram produces two dimensional images. More advanced techniques recently grabbing interest of researches producing three dimensional images of the tissue, such as MRI. A novel 3D imaging modality is Ultra-wideband (UWB) microwave imaging. Apart from producing three dimensional images, the UWB is based on a contrast in electrical properties of the tissues, and can detect malignant breast tumors even in “dense breast” situations. The UWB involves formation of a spatial image of scattered microwave energy, and to identify the presence and location of malignant lesions from scattering signatures thereof. There are different types of UWB imaging systems such as Hemispherical and cylindrical antenna arrays radar-based and tomography-based microwave breast imaging systems. Arrangement of antennas and configuration of the networking thereof have a large emphasis on the transmission of the microwave energies to the tissue and quality of imaging thereof.

The Ultra-wideband (UWB) microwave imaging technique involves transmitting microwave signals from antenna to the breast tissues and receiving the signal back therefrom. Such a technique involves formation of a spatial image of scattered microwave energy, and identification of the presence and location of malignant lesions from scattering signatures thereof. The conventional UWB systems involve very large arrays of antennas which may be configured to contact the tissue through an intermediate medium. FIG. 1 illustrates a device 100 for breast cancer detection using large array of antennas in the microwave frequency range Ultra High Frequency (UFO) Large Array antennas. The array antennas 102 may be placed on a radome structure that is made up of a material to improve matching of the signal transmitted or received by the antenna into the breast. The electromagnetic signals received by the antennas 102 are directed towards the breast skin 104 which then penetrates the skin to enter fat tissues 106 and fat-grandular tissues 108. However, the position and arrangement of antennas 102 of the device 100 suffer major drawbacks in generating high resolution 3D electromagnetic images of the breast tissues. The signal reaching the skin suffers from strong mismatch in electromagnetic properties which results in reflection of electromagnetic signals from the skin 104 and poor penetration. Due to further mismatch of dielectric constant between the skin (˜30) 104 and the fat tissues 106, reflections happen from the skin-fat boundary. In addition, an imperfect fit of the breast may generate a number of air bubbles between a cup (a housing that covers the breasts undergoing imaging) of the device 100 and the breasts, which may again affect the images of the tissues 104 and 106.

In light of the above limitations, it is desirable to have an imaging system which has antennas arranged such that there is a minimal reflection of electromagnetic signals from the skin-fat boundary and reduced air bubbles between the cup and the breasts. The imaging system of the present invention described herein comes to address this need.

### SUMMARY OF THE EMBODIMENTS

According to one aspect of the invention, an electromagnetic device for imaging a body tissue is disclosed. The device includes a plurality of flat circuit boards, wherein the plurality of the flat circuit boards are hinged together. A plurality of antennas are mounted on each of the flat circuit boards. Preferably, at least some of the antennas are dual-polarization antennas; for example, comprising two antennas that are arranged in orthogonal configuration forming a cruciform-antenna-pair. The arrangement of the flat circuit boards and the antennas form a tight array such that skin of the body tissue is in close contact with the antennas, allowing the dielectric constant of the antennas to match with that of the skin of the body tissue.

In another aspect of the invention, an imaging device for imaging the breasts for cancer diagnosis is disclosed. The imaging device includes a plurality of antennas placed on a plurality of flat boards. The antennas are arranged to form a polyhedral cup against which the breast is pressed. Each of the flat boards further includes one or more circuit boards, a plurality of these flat boards thereof are configured to hinge together. The circuit boards further comprise RF transceiver RFICs attached to said antennas. Preferably these are multichannel transceiver RFIC, such as Vayyar's VYR2401 (“Octopus”) or VYR7201 (“Centipede”) RFICs. Each RFIC may attach to antennas on a single board or to antennas on several adjacent boards. The RFICs preferably comprise signal acquisition and signal processing means. The RFICs on multiple boards may be configured to be networked together. Examples of methods to network multiple multichannel transceiver RFIC modules to form a larger multichannel transceiver system are described in a patent application WO2016051406A1. In the embodiment, a seam is provided between two adjacent circuit boards which create natural channels through which air may flow, thereby reducing air bubbles between the device cup and the breasts/tissue. The antennas are arranged such that the antennas are in almost direct contact with the skin of the breast with minimal intermediate medium, allowing the antennas having dielectric constant matched with that of the tissue or skin. Optionally, an intermediate medium of thin plastic insulator may be introduced for safety reasons or the like. The matching of dielectric constants reduces reflections from skin-fat boundary.

In yet another embodiment, an imaging system includes an imaging device, a cup, a gasket, a seal, and a handle. The imaging device further includes a plurality of antennas placed on a plurality of flat boards. The cup defines a housing to cover the breasts during the procedure of imaging thereof and mounted over the imaging device. The gasket is positioned on the cup, thereby providing a tight fit between the seal and the cup. The seal provides an ergonomic grip on the tissue undergoing imaging. The system also includes a handle disposed on outer side of the device which can be rotated to attach the device onto the breast.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the various selected embodiments may be put into practice. In the accompanying drawings:

FIG. 1 illustrates a schematic view of a prior art depicting a large array based imaging system;

FIGS. 2A-C show a possible arrangement of tight array of antennas of an imaging device 200 forming a polyhedral cup shape;

FIG. 3 illustrates a schematic view of an imaging device 300 according to an embodiment of the present invention;

FIGS. 4A, 4B and 4C illustrate the detailed internal components of the electromagnetic imaging device 400;

FIG. 5A and 5B illustrate the system components of the imaging device 500; and FIGS. 5C-5E illustrate different configurations of the seal of the imaging device 500;

FIGS. 6A-6C illustrate nested arrangement of the plurality of antennas in tight array configurations;

FIG. 6D illustrates a folded configuration of the tight antenna array to form a polyhedral cup 604;

FIG. 6E illustrates the folding of antenna array in an exemplary embodiment of the present invention;

FIG. 6F illustrates another exemplary arrangement of the antennas on the PCB;

FIG. 7 illustrates the performance of cavity-backed antennas depending upon the dielectric constant of a target medium;

FIG. 8A illustrates a PCB (printed circuit board) cavity-backed dipole antenna 802 in a PCB 800;

FIG. 8B illustrates match performance comparison of 2-wing cavity-backed dipole antenna;

FIGS. 9A-9C illustrates different configurations—900A, 900B, 900C of the cavity-backed dipole and cross-dipole antennas in a PCB;

FIG. 10 illustrates a single-ended microstrip line 1002 beneath one of the dipole sides with a capacitive feed to the opposite side of the dipole used in one of the antennas of the pair thereof; and

FIG. 11 illustrates an absorbent material 1102 surrounding the cup of the imaging device.

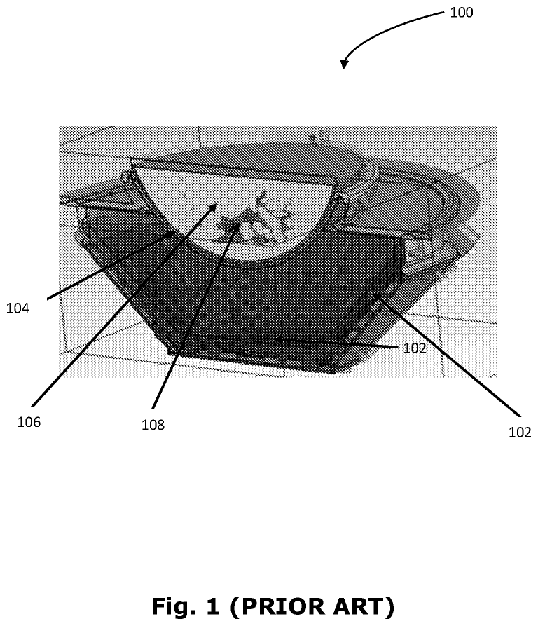

### DETAILED DESCRIPTION

Aspects of the present disclosure relate to system and methods for detecting breast cancer. In particular, the disclosure relates to radar based tight antenna array systems for breast cancer imaging and methods for performing mammographic scans therewith.

In various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally, or alternatively, the data processor may access a non-volatile storage, for example, a magnetic hard-disk, flash-drive, removable media or the like, for storing instructions and/or data.

It is particularly noted that the systems and methods of the disclosure herein may not be limited in its application to the details of construction and the arrangement of the components or methods set forth in the description or illustrated in the drawings and examples. The systems and methods of the disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting.

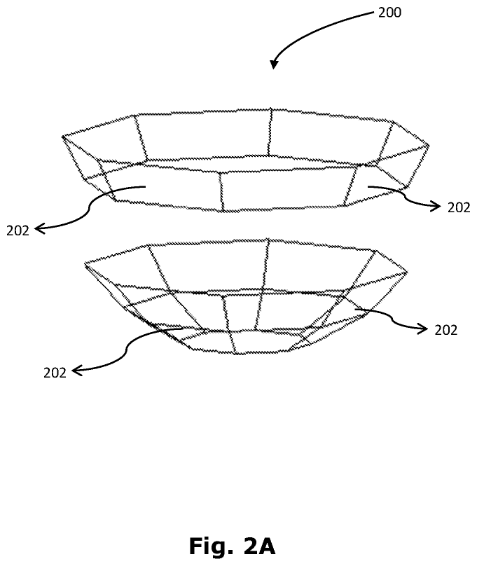

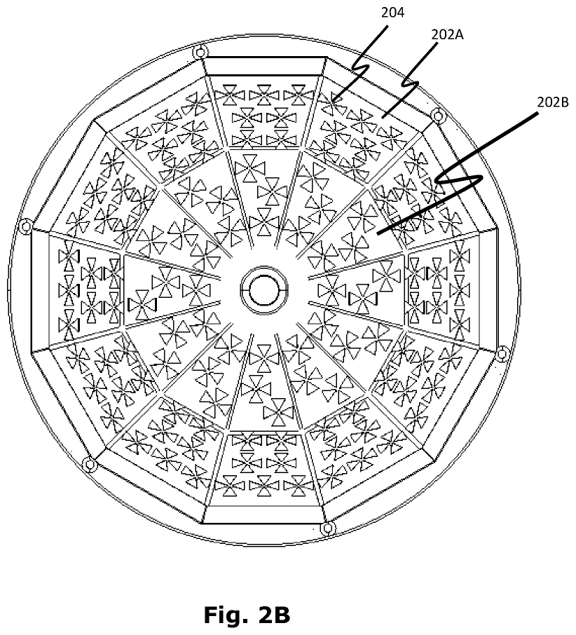

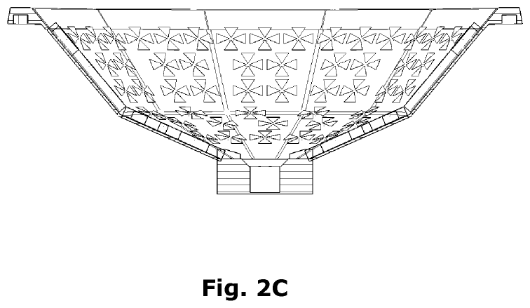

Reference is now made to FIG. 2A which illustrates schematic outer designs of an electromagnetic imaging device 200 forming a polyhedral cup shape. The device 200 includes a plurality of antennas arranged in a tight array. In a preferred embodiment of the present invention the antennas are placed on a plurality of flat circuit boards which are hinged together. In an aspect, there may be say twenty four flat boards 202 onto which antennas 204 may be positioned. The flat boards once hinged together may be folded to form the polyhedron cup 200. It is noted that the device 200 is very light weight. It should be clear to a person skilled in the art that the number of flat boards, number of antennas and weight measurement disclosed above are exemplary in nature and should not limit the scope of the invention.

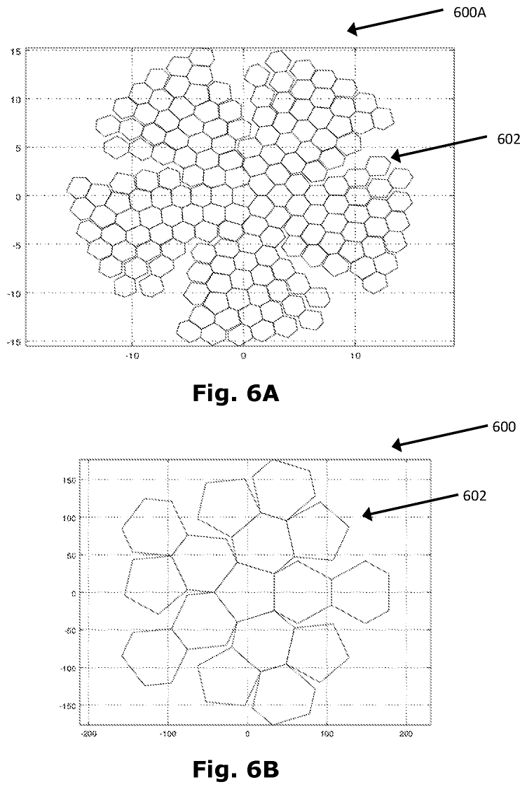

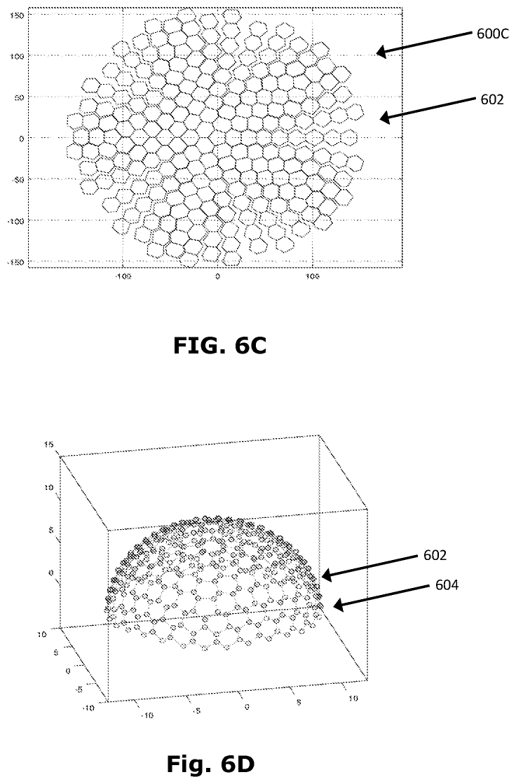

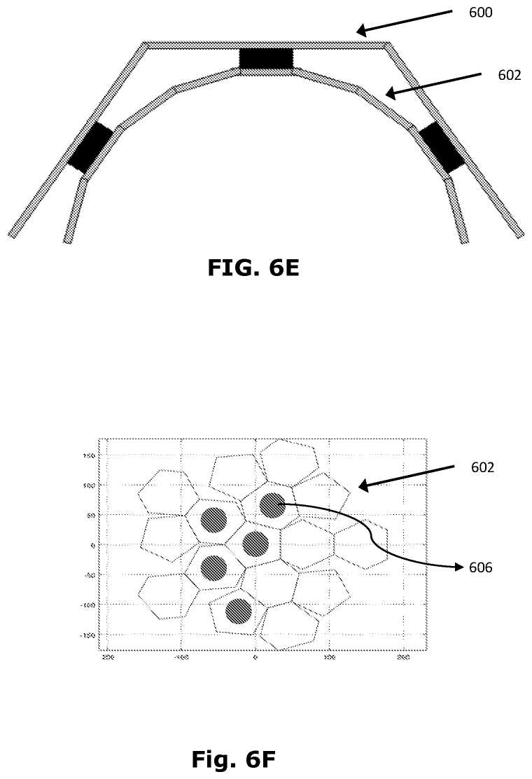

FIG. 2B is a top view and FIG. 2C is a side view of the polyhedral cup showing the arrangement flat board mounted antennas 204 upon a polyhedral cup formed from a first ring of twelve outer flat boards 202A and a second ring of twelve inner flat boards 202B. The antennas 204 are arranged in orthogonal pairs of dipole antennas each forming cruciform antenna-pairs to operate as a dual-polarization antenna. Each inner flat board 202B has three cruciform antenna-pairs and each outer flat board 202A has seven cruciform antenna-pairs such that two hundred and forty flat board mounted antennas are fitted onto the cup. It is noted that other geometries may be preferred as suit requirements. For example, utilization of polyhedral shape with larger number of surfaces facilitates tighter following of the body's contour. Examples of such arrangements are further illustrated in FIG. 6.

For example, a coarse polyhedron is a dodecahedron (12 pentagons) and the refined polyhedron has 252 (=12*21) faces. Each subarray has 21 antennas arranged as follows:

21=(1+5(layer1)+10(layer2)+5(part of layer3)) faces (1)

Such a subarray is configured to suit a single octopus for one polarization or 2 octopuses for dual polarization. The configuration has 126 antennas per hemisphere.

In another example, the coarse polyhedron is a dodecahedron (12 pentagons) and the refined polyhedron has 492 (=12*41) faces. Each subarray has 41 antennas arranged as follows:

41=(1+5(layer1)+10(layer2)+15(layer3)+10(part of layer4)) faces (2)

Such a subarray is configured to suit 2 octopuses per module. The configuration has 246 antennas per hemisphere.

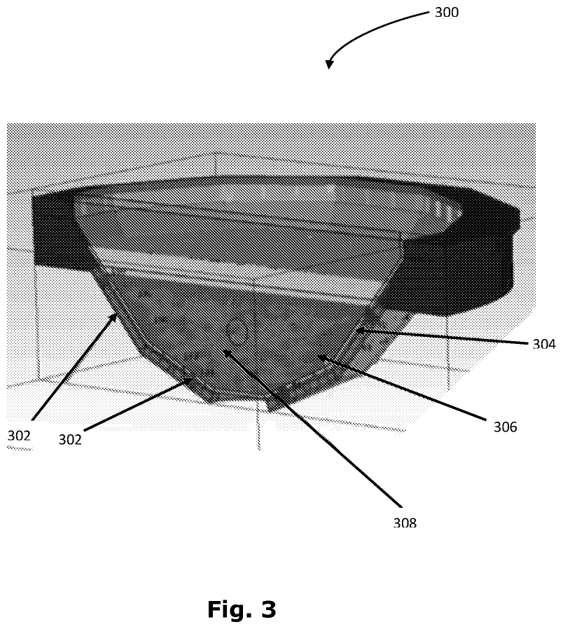

FIG. 3 illustrates a schematic view of the electromagnetic imaging device 300. The antennas 302 are arranged to form a polyhedron cup against which the breast skin 304 is pressed. The polyhedron cup 200 defines three-dimensional polytopes. The antennas 302 are arranged such that they may be in close or direct contact with the skin 304 with little or no intermediate medium, allowing the dielectric constant of the antennas 302 matched with that of the skin 304 and fat reducing reflection of electromagnetic signals from skin-fat boundary. The dielectric constant of the skin of the tissue is about 30. The reduced reflections of electromagnetic signals improve the 3D electromagnetic simulation imaging. Where appropriate, an intermediate medium of thin plastic insulator may be introduced to separate the skin from the cup as may be required for safety reasons or like.

The antennas 302 are hinged together tightly allowing minimal space between the two adjacent antennas 302. The space between antennas 302 may be occupied by fitting certain materials which may be configured to absorb extra wave energies that may have probability to penetrate into the tissue 306. Such a tight array of antennas 302 may form a Closed Faraday like cage. Examples of fitting material may include such as, but are not limited to, metals like copper, aluminum, and so on. Moreover, the arrangement of the antennas 302 in cross polarized array may be applicable for both low and high density Fibro-glandular tissues 308 thereof.

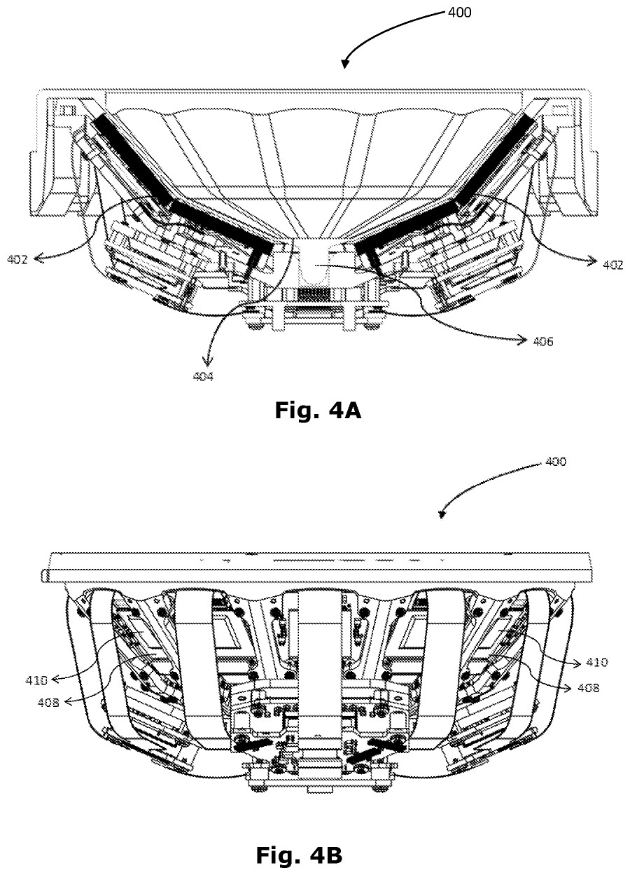

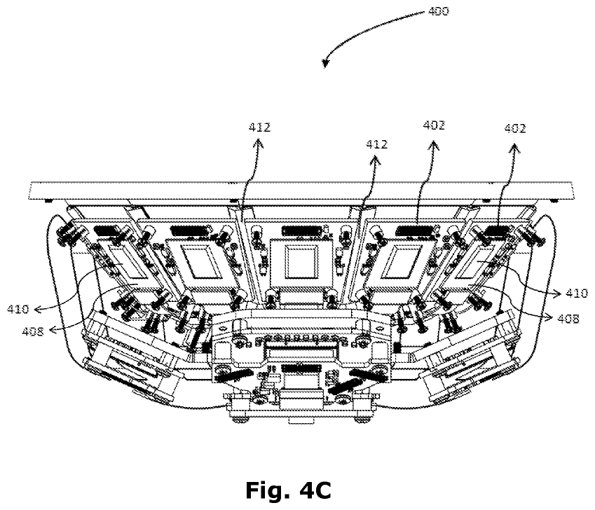

Referring to FIGS. 4A, 4B and 4C which illustrate the detailed views of the electromagnetic imaging device 400. The device 400 includes various antennas 402 hinged on various flat boards in a polyhedral shape. The device 400 also includes a gap or a hole for the breast nipple 406 to fit-in. This allows better attachment of the device 400 with the breast skin. The device 400 further includes various flat boards which includes a circuit board 408, a plurality thereof are configured to hinge together. Each of the circuit boards 408 has own RF transceiver RFIC 410 forming a multi-module system. The multi-module configuration of the system 400 improves performance and reduces cost. Exemplary Multi-chip module technologies which can be used includes, but not limited to, the IBM Bubble memory MCMs, Intel Pentium Pro, Pentium D Presler, Xeon Dempsey and Clovertown, Sony memory sticks and similar devices. All the chips 410 are configured to be networked together for ease of management and control. The multiple RF transceiver RFICs may be interconnected to form larger multichannel transceiver systems, for example using methods described in a patent application WO2016051406A1 which is incorporated herein by reference in its entirety. In the present embodiment, there may be seams 412 for example between two adjacent circuit boards 408 as shown in FIG. 4C or between the flat surfaces of the cup. The seams 412 create natural channels through which air may flow, thus allowing an evenly distributed low-pressure region, or partial vacuum, to be established over the surface of the breast, thereby reducing air bubbles between the device cup 300 and the breasts skin 304.

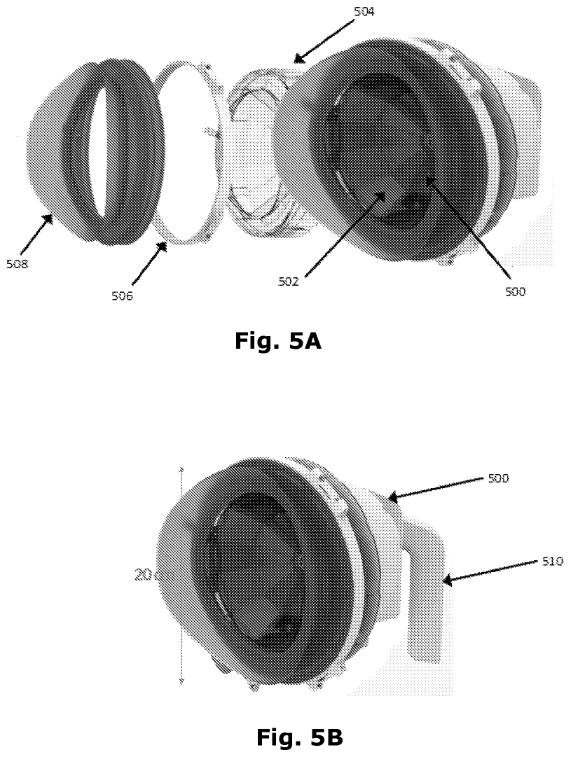

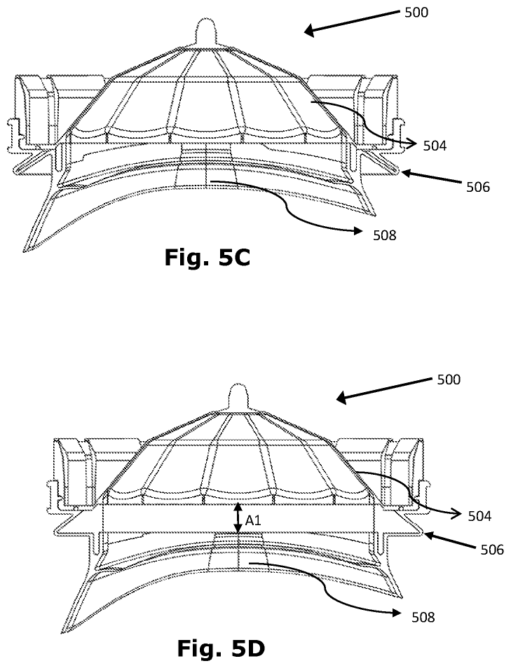

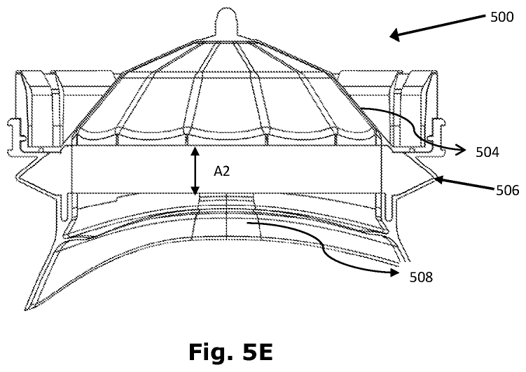

FIG. 5A and 5B illustrate the system components of the imaging device 500. The imaging device 500 includes a cup 504, a gasket 506 and a seal 508. The imaging device 500 further includes a plurality of antennas 502 placed on a plurality of flat boards. The antennas 502 are arranged to form a polyhedral cup against which the breast is pressed.

The cup 504 defines a housing to cover the breasts during the procedure of imaging and mounted over the imaging device 500. The cup 504 has dimensions such that any size of the breast of different females can be mounted thereon. The seal 508 provides an ergonomic grip on the breast undergoing imaging. In a preferred embodiment, the seal 508 is a silicon component which attaches to the breast skin using a vacuum pump device. The shape of the seal 508 is such configured for tight fitting on the breast. The use of flexible silicon gasket for the seal 508 allows smooth and proper attachment to the breast without causing any harm to the breast skin. Alternatively, certain polymers, such as thermoplastic elastomer, thermoplastic rubber, and polyvinyl chloride, possess qualities similar to those of silicon and can be used for seal 508. The gasket 506 connects the seal 508 with the cup 504 ensuring perfect fitting. The gasket 506 is positioned on the cup 504, thereby providing a tight fit between the seal 508 and the cup 504. The gasket 506 is configured to be placed on the cup 504 and rotated, thereby locking the cup 504 with the gasket 506. The cup 504 is fitted to the device 500 which is then attached to the breast through the seal 508. Hence the arrangement of the system is such that the device 500 mounts on the cup 504, the gasket 506 is placed over the cup 504 and further the seal 508 mounts over the cup 504 through the gasket 506 as well as houses the breast undergoing imaging.

The device 500 also includes a handle 510 disposed on outer side of the device 500 as shown in FIG. 5B. The handle 510 helps to rotate the device 500 for fitting the device 500 perfectly on the breast. In an embodiment, the handle 510 may be rotated at 30 degrees in clockwise direction to fit the device 500 with the breast. The use of handle 510 also allows the device 500 to be compatibly fitted on various size breasts. The seal 508 is further configured to expand as per requirements of the size of the tissue, as shown in FIGS. 5C-5E. As shown in FIG. 5C, the cup 504 surrounded by the gasket 506 includes the seal 508 disposed in perpendicular alignment to the cup 504. As the seal 508 expands by “A1” shown in FIG. 5D, the gasket 506 also expands horizontally along with that of the seal 508 and expands to a complete extent “A2” as shown in FIG. 5E. The expanded seal 508 and the expanded gasket 506 automatically enlarge the cup 504 to accommodate larger size of the breast. The material of the gasket 506 is such that the gasket 506 is expandable and can be made from same materials as that of the seal 508. In some aspects, the gasket 506 may be made from solid contoured frame thin elements which may be opened up as the seal 508 expands.

The device 500 is configured such that the device 500 is lightweight and do not bear much weight despite of accommodating large number of antennas 302. Lightweight feature of the device 500 thereof may not put extensive weight on the breast, providing comfort to the females undergoing examination. The handle 510 may provide handheld feature which may ease handling of the device 500.

In some aspects, the array of the antennas 602 may be arranged in different spatial arrangements as illustrated in FIGS. 6A-6C. Covering the examined region with a polyhedron having larger number of faces allows for smoother following of the inner volume, improving the fit between the body and the antenna array. As illustrated in FIG. 6A, the arrangement 600A of the array of the antennas 602 depicts a tight array of the antennas such that the antennas 602 occupy multiple faces of the flat boards. Multiple boards comprising the antenna array may be produced as a flat printed circuit board, which is later folded into a spatial shape as illustrated in FIG. 6D. To facilitate the folding, the PCB sections comprising the facets of the polyhedron are preferably interconnected by flexible-PCB sections. These flexible interconnects may convey RF signals, as well as comprise a shielding ground layer. The flat circuit board comprises the multiple boards designed according to the unfolding of the spatial shape.

The transceiver RFICs may be mounted directly on the antenna board, or on a daughterboard serving multiple facets of the polyhedron. An example of such arrangement is exemplified in FIG. 6E. The boards 600 comprise the transceivers and form a coarse polyhedron. The boards 602 form a fine-grained polyhedron for better following of body's surface. In such an example, the coarse-grained polyhedron may have an unfolding resembling FIG. 6B, while the fine-grained polyhedron may have an unfolding resembling FIG. 6C.

Exemplary designs of covering a sphere are based on combinations of pentagons and hexagons. For example, dodecahedron comprises 12 pentagons, while the iconic “football” design comprises 12 pentagons and 30 hexagons. Further refinement is possible by designs based on placing face centers in the 12 corners of an icosahedron and subdividing the 20 triangular facets according to a triangular grid to form additional face centers on a sphere.

Each of the faces of the flat boards requires antennas 602 to have their own polarization. Further as shown in FIG. 6B, the arrangement 600B shows a nested arrangement of the antennas 602, while FIG. 6C shows a tight array of the nested arrangement 600C of the nested antennas 602.

There are many benefits of the tight array of the antennas such as including but are not limited to no airgaps between the tissue and the antennas, thereby no stray waves travel around and create high-delay leakage. Consequently, no coupling losses while using antenna-air+air-medium; and no loss of angular coverage−Snell's law for air→body interface. In addition, the antenna array as embodied hereinabove allows matching of the dielectric medium even in cases of shorter wavelength. Therefore, the antenna array facilitates less leakage from array boundaries, and simpler production by having interconnects being part of the flat boards.

In the above different spatial arrangements of the antennas 602, the antennas 602 may be configured to be embedded on the flat boards. Such flatboards may further attain folded configuration to form a polyhedral cup 604 as shown in FIG. 6D. For folding a tight array of antennas into a polyhedral cup, fit a circular footprint of antennas for arbitrary orientation and polarization selection. The embedded or mounted antennas are assembled before the folding of the array to its spatial shape. The antenna array is then folded as illustrated in FIG. 6E. The dense polyhedra are useful for antenna arrays that follow closely the spherical inner surface of the cup.

In another aspect, the antennas 602 may be mounted on the flat boards. Preferably, all boards have a similar-sized shape. To allow the boards to accommodate antennas that can be oriented in an arbitrary polarization or orientation, it is preferable that the antennas occupy a circular footprint. As shown in FIG. 6F, each of the antennas 602 fits a circle 606 enabling the antennas 602 to be rotated to an arbitrarily angle, while fitting onto the PCB facet.

In an exemplary embodiment of nested arrangement of 21-antenna module, 12 such modules cover a sphere and 6 modules cover a hemisphere. For 10 cm radius sphere, each face can accommodate a circle of 2 cm diameter for the antenna. For a hemisphere of 1000 cc volume, the radius is 7.8 cm. In another exemplary embodiment of nested arrangement of 41-antenna modules, and 492 spheres, fora 10 cm radius sphere, each face can accommodate a circle of 1.4 cm diameter for an antenna.

In an exemplary embodiment, the distance of corners vs. center of a nested polyhedral arrangement may be as follows:
For Dodecahedron (12 pentagons) the corners are 1.25R from the center
   For R=10 cm translates into 2.5 cm corner depth.
   The excess distance (corner depth) reduces ˜inversely to number of faces
   For 92 (12 pentagons, 80 hexagons)—4 mm
   For 252 faces (12 pentagons, 240 hexagons)—1 mm
   For 492 faces (12 pentagons, 240 hexagons)—0.5 mm
Face area is ˜ sphere surface area/#faces (1256 cm^2 for R=10 cm)
   For 252 face area is ˜5 cm^2
   For 492 face area is ˜2.5 cm^2

The above exemplary dimensions illustrated hereinabove, should be contemplated that the dimensions are not limited in scope, however are illustrated hereinabove for enabling the ordinary persons skilled in the art to understand the present invention.

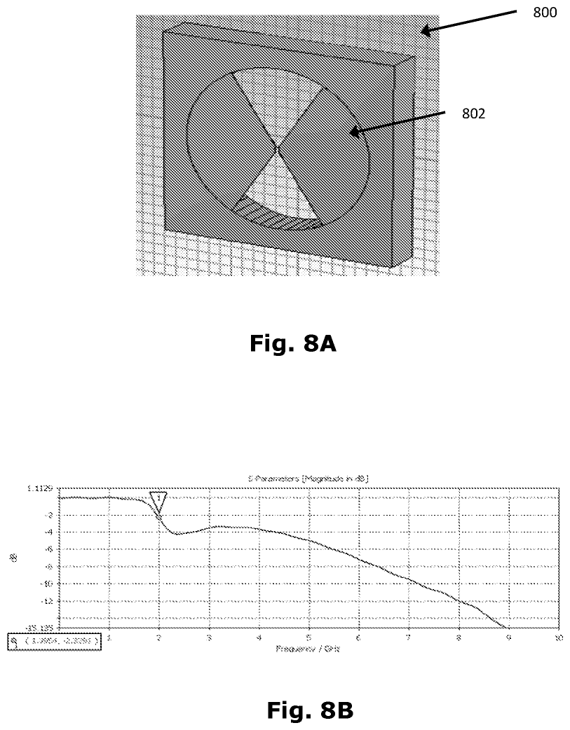

FIG. 8A illustrates a PCB (printed circuit board) cavity-backed dipole antenna 802 created by making a cavity in a PCB 800. Such pairs can have different number of antennas, and configurations thereof. As shown in FIG. 8A, two antennas or wings face each other and are shorted slot-dipole. The exemplary arrangement of cavity-backed antennas 802 shown in FIG. 8A can have a radius of 8 mm. At eps=10 of the body medium and eps=4 of the PCB flatboard, such wings/antennas show reasonable matching above 2 GHz as shown in graph of FIG. 8B. The cavity preferably has an approximately circular shape, to allow arbitrary orientation.

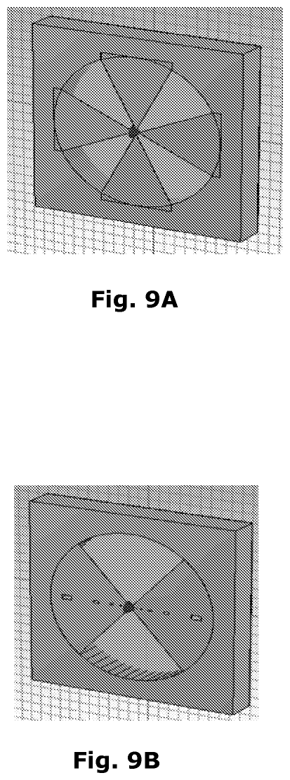

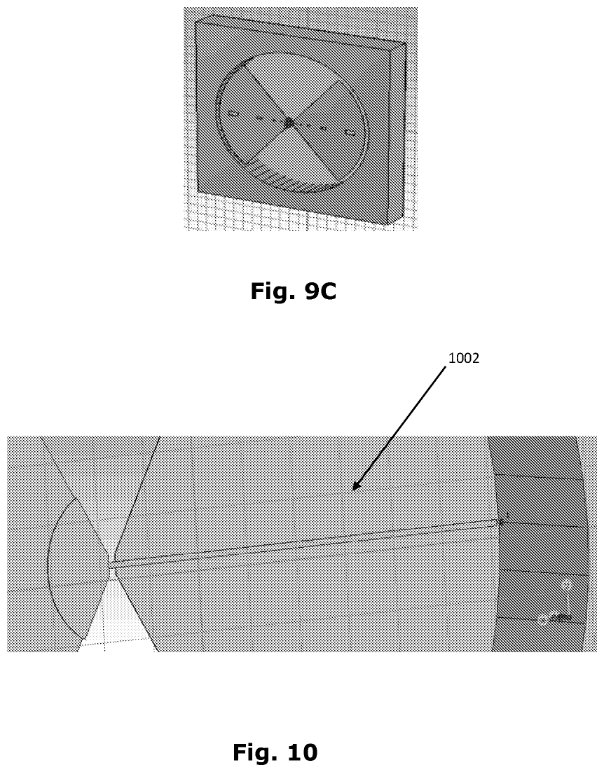

In some other aspects, four or more wings can form a cavity-backed cross-dipole antenna to form cruciform-antenna arrangements as shown in FIG. 9A. In yet some other aspects, the antennas can be shorted as shown in FIG. 9B, and open-ended 904 as shown in FIG. 9C.

The following modifications are possible on the cavity antenna wings:
Open-ended vs. shorted vs. edge-loaded
Zigzagged, meshed, dual-zigzag,
2 or 4 wings (for dual polarization option)
Ring-loaded
Varying the substrate thickness

In an exemplary embodiment, the following advantages are achieved with Open-ended dipole cavity-backed antennas as compared to End-loaded dipole cavity-backed antennas:
Improves out-of-band (low freq) matching
Reduces efficiency (reflected wave not reused for resonance)
Increases high-frequency cut-off only marginally

The board thickness, and correspondingly the depth of the cavity backing the dipole(s), has substantial impact on radiation efficiency. For example, increasing the PCB thickness from the commonplace 1.6 mm to 3 mm improves the radiation efficiency substantially. As the depth of the cavity increases further, there is a less pronounced difference between the radiation efficiencies of the antennas.

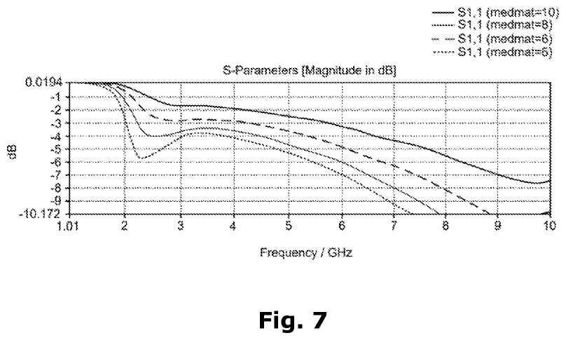

FIG. 7 illustrates the performance of the proposed cavity-backed antennas depending upon the target medium. The antennas show an acceptable matching starting at 2-2.2 GHz at medium eps=10. At epsilon below 10 the performance of the cavity antennas does not fall apart although the matching gets gradually worse and the lower cutoff increases a bit.

In some embodiments, at least one of the wings of the dipole-antenna arrangement can include a single-ended microstrip line 1002 as shown in FIG. 10, with the wing serving as a ground plane. Such a line 1002 acts as a as a balun for feeding the dipole. In the opposite wing of the dipole, a printed matching capacitor may be disposed under thereof. In the cruciform-antenna arrangement, each of the two dipoles of the cross-dipole has its own feed line.

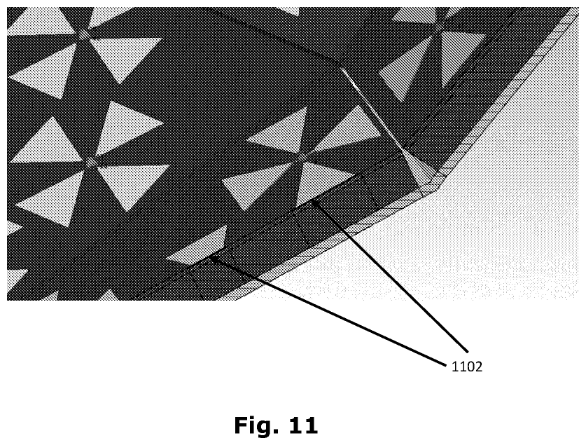

In some embodiments, an absorbent material 1102 surrounds the antennas to improve transmission of electromagnetic signals into the body, while attenuating surface waves creeping along the periphery of the body, as shown in FIG. 11. Examples of absorbent material 1102 may include, but are not limited to, carbon-loaded foam, carbon-loaded epoxy resin, a dielectric material with a high loss factor, and so on. Such absorbent material may be disposed in a form of coating surrounding the antennas such that the electromagnetic waves transmitted can be absorbed to avoid the losses. In some aspects, the absorbent material can be in the form of powder, viscous solution or paste which dries up after a shorter period of time. In an embodiment, the material may remain in a liquid or viscous form and be contained behind a thin membrane to avoid spillage.

Hence, the device 500 (or 300 or 400) thereof may have advantageous effects such that the antennas 302 are arranged to be in direct contact with the skin 304, matching the dielectric constant of the skin 304 and reducing reflections from skin-fat boundary of the breast. The seams 412 may cause reduction in air bubbles between the device 500 and the breast, providing better images. Moreover, the arrangement of the antennas 302 in cross polarized array may be applicable for both low and high density Fibro-glandular tissues thereof.

### Technical Notes

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%. The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described hereinabove as well as variations and modifications thereof, which would occur to persons skilled in the art upon reading the foregoing description.

1. An electromagnetic device 400 for imaging a body tissue, the device comprising:
a plurality of flat circuit boards 408, wherein the plurality of the flat circuit boards are hinged together; and
a plurality of antennas 402 mounted on each of the flat circuit boards, at least two antennas of the plurality of antennas form a dual-polarization antenna;
wherein the arrangement of the flat circuit boards and the antennas form a tight array such that skin of the body tissue is in close contact with the antennas,
and wherein the antennas are configured to radiate into a medium having a dielectric constant of a body tissue.

2. The device of claim 1, wherein the plurality of flat circuit boards are hinged together to form a polyhedral cup configuration.

3. The device of claim 2, wherein the polyhedral cup configuration comprises one or more rings 202A, 202B having flat circuit boards mounted on each of the rings.

4. The device of claim 1, wherein each of the plurality of the flat circuit boards has an outer surface and an inner surface, wherein a varying number of antennas are disposed on each surface of the flat boards.

5. The device of claim 1, wherein each of the flat circuit boards comprise a radio frequency transceiver RFIC 410 for controlling the functioning of antennas mounted on the circuit board.

6. The device of claim 5, wherein the radio frequency transceiver RFICs of each of the flat circuit boards may be networked together.

7. The device of claim 1 further comprises a seam 412 between two adjacent circuit boards for creating natural channels through which air may flow, thereby reducing air bubbles between the device and the body tissue.

8. The device of claim 1, wherein the matching of dielectric constants of the antenna and the skin of the body tissue reduces reflections from skin-fat boundary.

9. The device of claim 2, wherein an expandable seal 508 surrounds the polyhedral cup, customizing the cup for accommodating multiple sizes of the body tissue.

10. The device of claim 1, wherein an absorbent material 1102 surrounds the antennas to improve transmission of electromagnetic signals.

11. The device of claim 1, wherein the plurality of antennas are assembled in an array and folded to form a polyhedral cup 604 shape.

12. The device of claim 1, wherein one or more antennas of the plurality of antennas are formed as cavity-backed dipole antennas 802 on the flat circuit boards.

13. The device of claim 12, wherein at least two cavity-backed dipole antennas are arranged in an orthogonal configuration forming a cruciform-antenna-pair.

14. The device of claim 12, wherein the dipole antennas are in one or more of an open-ended, shorted and edge-loaded configuration.

15. The device of claim 12, wherein the cavity-backed dipole antennas are in one or more of a zigzagged, meshed and dual-zigzag configuration.

16. The device of claim 12, wherein the cavity-backed dipole antennas are in a 2-wing or a 4-wing configuration.

17. The device of claim 1, wherein the body tissue is a breast.

18. The device of claim 1, wherein the device is configured for imaging a breast for diagnosing breast cancer.

19. The device of claim 2, wherein said plurality of flat circuit boards hinged together to form a polyhedral cup configuration comprises at least six boards.

20. An electromagnetic imaging device 400 for diagnosing breast cancer, the device comprising:
a plurality of flat circuit boards 408, wherein the plurality of the flat boards are hinged together to form a polyhedron cup configuration;
a plurality of antennas 402 mounted on each of the flat circuit boards, at least two antennas of the plurality of antennas are arranged in orthogonal configuration forming a dual polarization antenna;
characterized in that the polyhedron cup accommodates a breast in a tight fit;
wherein the arrangement of the flat circuit boards and the antennas form a tight array such that skin of the breast is in close contact with the antennas, allowing the dielectric constant of the antennas to match with that of the skin of the breast.

21-51. (canceled)
